(19) United States
(12) Patent Application Publication
(10) Pub. No.: US 2019/0343402 A1
(43) Pub. Date: Nov. 14, 2019

(54) DEVICE FOR LOWERING THE RISK OF CANCER AND/OR THE RISK OF A CANCER RELAPSE AND/OR OTHER DISEASES

(71) Applicant: movival GmbH, Achern (DE)

(72) Inventors: Alexander GEHRING, Achern (DE); Thomas WIDMANN, Buehl (DE)

(73) Assignee: movival GmbH, Achern (DE)

(21) Appl. No.: 16/478,561

(22) PCT Filed: Feb. 1, 2018
(86) PCT No.: PCT/DE2018/000018

§ 371 Date: Jul. 17, 2019

(30) Foreign Application Priority Data
Feb. 1, 2017 (DE) 10 2017 101 967.7
Apr. 28, 2017 (DE) 10 2017 109 272.2

(51) Int. Cl. A61B 5/0205; A61B 5/11; A61B 5/00; A61B 8/02

(52) U.S. Cl. CPC A61B 5/0205; A61B 5/1118; A61B 5/742; A61B 5/6898; A61B 5/681; A61B 5/0022; A61B 8/02

(57) ABSTRACT

A device for lowering the risk of cancer and/or the risk of a cancer relapse and/or other diseases includes an internet portal, for detecting a user's physical activities, which are carried out using a connected database and corresponding algorithms relating to a recommended daily dose of physical movement and on the basis of which recommendations to the user for additional physical activities are automatically generated and transmitted to the user so that the user is capable of performing the daily or weekly user-individual physical activities that are suitable for significantly lowering the risk of cancer or the risk of a cancer relapse. The system is designed as a self-learning unit designed such that the recommendations which are vocalized by the device are adapted constantly and are improved while taking into consideration the accumulated data.

The invention relates to a device for lowering the risk of cancer and/or the risk of a cancer relapse and/or other diseases. In this regard, in medicine, cancer is understood to be malignant tissue neoplasms or a malignant tumor. In general language usage, cancer is understood to be a collective term for a great number of related diseases, in which body cells can grow and divide in uncontrolled manner, displace healthy tissue and destroy it. Cancer diseases are attributable to different triggers, such as unhealthy eating, smoking or strong sun irradiation or the effects of natural or artificial toxins. In this regard, theoretically any organ of the human body can be affected by cancer. In this regard, the frequency of most cancer diseases clearly increases with age. For this reason, cancer is sometimes also viewed as a disease of old age of cell growth. In this regard, it has been possible up to now to minimize the risk of cancer by means of early detection of cancer. Diseases that can also be fought against using the device according to the invention include, in particular, stroke (apoplexy), cardiac infarction (cardio diseases), narrowing of the arteries/blood vessels, osteoporosis, diabetes, COPD or asthma and chronic back pain.

In particular with regard to cancer, which occurs more frequently with increasing age, new knowledge has recently been acquired to the effect that an increased susceptibility to cancer occurs due to age-related shortening of what are called telomeres, in other words the ends of the chromosomes, which consist of repetitive DNA and associated proteins, because in this way, the risk of chromosome breaks and thereby of events that trigger cancer increases.

According to comparatively most recent knowledge from medical research, the enzyme telomerase (an RNA protein complex with reverse transcriptase activity) can balance out shortening of the telomeres again, at least in part. In this regard, the enzyme telomerase can be activated by means of physical activity. However, this activation must be repeated every 24 hours. This permits determination of a prevention approach for cancer prophylaxis or cancer relapse prophylaxis. The invention to be explained below is based on this knowledge.

According to the current claim 1, the invention relates to a device for lowering the risk of cancer and/or the risk of a cancer relapse and/or other diseases. Advantageous embodiments of the device according to the invention can be found in the dependent claims 2-18.

First of all, the device according to the invention comprises a detection device for physical activity and/or an input device for manual input of physical activities performed by the respective users, i.e. the device according to the invention. Both the aforementioned detection devices and the input devices are connected with a server with which a database is connected. In the end result, the server can usually be reached by way of an Internet portal connected with the devices. The physical activities reported by the users, in the way mentioned, are converted to what are called Metabolic Equivalents, abbreviated as METs, using a conversion formula stored in the server, in connection with a concordance table, recorded, and stored as METs or MET points. The Metabolic Equivalent makes it possible to make the energy consumption of the human body connected with physical activities comparable. It stands for the description of the metabolic rate of a human being, with reference to the resting rate in relation to his/her body weight. One MET corresponds to the rate of 3.5 ml oxygen per kilogram of body weight per minute in men and of 3.15 ml oxygen per kilogram of body weight per minute in women. To state it differently, 1 MET stands for an energy consumption of 4.2 kJ, which corresponds to one kilo-calorie per hour. In both cases, this dimensional unit defines the resting rate of the human body, in other words as long as it is not moving.

According to the invention, the metabolic equivalents related to the movement activities reported by the users are stored in the database, for example 3.5 MET for 1 hour of mowing the lawn or 7 MET for 1 hour of jogging. For regular activation of the enzyme telomerase, a value of 27 MET should be reached per week by an average user, in other words approximately 4 MET per day so as to ensure regular activation of the enzyme with the aforementioned 24-hour period. On the whole, in this connection the principle therefore applies that even with a little more movement, easy improvement with regard to the risk of a cancer relapse or the risk of cancer can be achieved, and that a decisive improvement in this area can already be achieved by activity that goes slightly beyond this movement. The lowering of the risk of the occurrence of cancer or the risk of a cancer relapse that can be achieved by means of more activity therefore does not have a linear progression, but instead, additional activity is rewarded by an above-proportional improvement in the person's own prognosis, with regard to the likelihood of becoming ill with cancer or with a cancer relapse.

The solution according to the invention therefore provides that as a reaction to the physical activities reported by way of the detection device or the input device, the user is provided with a graphic and/or numeric display, for example within the scope of an indication of percentage, that puts the physical activities performed into relation with the movement dose recommended for the respective user, against the background of the above considerations. Within the scope of the graphic display, the user can be optically shown whether or not and to what extent he/she lies above the movement dose recommended for him/her, in other words more or less above or below the value of 4 MET per day.

In this regard, it is advantageous if the aforementioned input device is a conventional smart phone, a tablet or a computer or some other device capable of Internet connectivity, which is enabled, by means of a mobile app, to enter into data exchange with the server of the device according to the invention.

In a further embodiment, the device according to the invention is connected with a response system connected with the server of the device, which system gives the user feedback with regard to the physical activities reported or recorded by him/her, in other words praises him/her, for example, or motivates him/her to undertake additional activities so as to reach his/her goal for the day. For this purpose, suggestions concerning physical activity that are stored in the database can be accessed on a user-individualized basis; in other words, a physical activity can be suggested, for example, that precisely corresponds to the movement dose that is still missing. For example, taking a walk for half an hour can be suggested. In an advantageous embodiment, the system can evaluate data from outside the system, in this regard, in other words take the current weather report into consideration, for example, and suggest indoor or outdoor activities accordingly. In a concrete embodiment, the corresponding suggestions and feedback are transmitted to the detection devices and/or input devices for physical activities that are connected with the server in terms of data.

In an advantageous further development, a user-individualized movement journal is drawn up using the database connected with the server, on the basis of the physical activities reported by the user, which journal can be called up by the respective user at any time, by way of the detection devices or input devices connected with the server of the device. The user can therefore call up the results of his/her activities or also the effects of possibly reduced activity on the terminal operated by him/her, at any time. This also represents a contribution to motivating the users of the system.

A further advantage of the device according to the invention consists in that ultimately, a profile of the physical activity of the user is formed by means of regular reporting of the physical activities of the user and their evaluation. In connection with an additional function that can be selected, these data can be made available to selected doctors working with the device according to the invention, with the approval of the user, which doctors can issue supplemental recommendations on the basis of the data available to them as a result, if applicable also taking into consideration a history of the disease known to the doctor.

In a further embodiment of the apparatus according to the invention, the users can also enter any existing physical restrictions or other restrictions into their user profile, which can then be called up by way of the database and taken into consideration in the aforementioned recommendations of the doctor as mentioned above or in the evaluation of the physical activities, for example. Furthermore, however, these user data can also be taken into consideration in the suggestions regarding physical activity that are made by the device according to the invention, so that they can be realistically implemented by the respective user. In a further embodiment, the device according to the invention suggests a user-individualized movement plan, which takes into consideration not only the preferences but also the restrictions or other further circumstances, wherein these movement plans can also be called up at any time, once again, by way of the addressed portal, by means of terminals operated by the user.

Furthermore, it has proven to be advantageous if the users of the system can communicate with one another by way of the portal connected with the server, wherein this is also possible in the form of bidirectional communication, such as by way of discussion forums set up on the portal. In the end result, the users can motivate each other, for example, to achieve their goals or default values with regard to physical activities, or can simply make arrangements to become physically active together.

As explained above, the device according to the invention is based on strict implementation of medical and scientific knowledge, wherein this knowledge has now been established, in the meantime, on a comparatively assured basis of fact, but these facts are constantly being developed further and improved on the basis of further and more recent data collection, wherein last but not least, the database used within the scope of this device to record the physical activities, in connection with the user profile, in other words the disease history of the user, ultimately enters further data into the system, which data in turn are drawn up, with the user's approval, preferably in anonymous form, by means of comparison and optimization algorithms connected with the database and the server, and evaluated for optimization of the device according to the invention, in such a manner that the movement plans or proposals regarding physical activity suggested to the individual users are adapted to the knowledge obtained in this way. If understood correctly, the device according to the invention is therefore a self-learning system. The basis of the self-learning system is existing scientific/medical data, for example from patient studies. Since it has been shown that in the case of medical patient studies, the types of cancer that occur most frequently are predominantly studied, this results in a negative selection of rare cancer diseases. In any case, the approach described here would be suitable for aligning the existing medical database with the data obtained within the scope of utilization of the device according to the invention. Based on the self-learning nature of the apparatus, a more precise, i.e. better recommendation regarding the level of activity can be issued to the users of the device according to the invention in the case of an improved data situation from the data pool of the device. What is expected here is that in particular in the case of the types of cancer that do not occur most frequently, the device can issue even better individualized recommendations.

In a further embodiment of this approach, user data from outside the system, for example from medical studies or new medical knowledge, are also constantly entered into the device as calculation approaches, particularly into the comparison and optimization algorithms, and this in turn results in improved suggestions regarding physical activity and optimized movement plans, in user-individual manner.

In a concrete embodiment, the device according to the invention works together with a pulse measurement instrument that works with ultrasound detection of the heart rate.

Ideally, the pulse measurement instrument is implemented as a pulse watch, which can be worn on the wrist of the user of the device according to the invention, in simple manner.

In this regard, the pulse watch comprises an ultrasound head for emission of ultrasonic waves, wherein the pulse watch can be fastened to the wrist of the respective user of the pulse watch, with the interposition of a coupling cushion that follows the underside of the pulse watch. In this connection, the problem exists that as little air as possible must be present at the boundary surface of the ultrasound probe, in other words at the sound head, and the skin of the user, so as to prevent possible sound reflections that distort or impair the measurement result. For this purpose, an ultrasound medium should be used that is disposed on the underside of the pulse measurement device and, at the same time, lies on the skin of the user in comfortable manner. This dual function is fulfilled by the coupling cushion on the underside of the pulse watch that was mentioned above.

In a concrete embodiment, the coupling cushion comprises a gel body, wherein the gel body is produced on the basis of reaction products of polyisocyanates and polyols.

In an advantageous embodiment, the gel body is sheathed with an elastic and/or stretchable sheath, preferably composed of a polymer film. Such complete sheathing of the gel body is required if only for hygiene reasons. Use of elastic or stretchable sheathing materials contributes to a pressure-distributing effect and accordingly increases wearing comfort.

In a further embodiment of the pulse watch, the ultrasound head itself is integrated into the gel body.

In yet another further development, the gel body is releasably connected with the underside of the pulse watch, with the interposition of a hard plastic, so that the pulse watch can also be worn as a conventional watch, without the gel body, for example, and the gel body is only set onto the underside when the pulse watch is used for sports purposes, for example. Furthermore, in this manner the gel body can be easily replaced, for example for hygiene reasons or because the integrated ultrasound head has given up its function.

Once again in a concrete embodiment, the gel body is welded into a hard plastic mantle, with the integrated ultrasound head, which mantle is releasably connected with the underside of the pulse watch, as has been mentioned.

In the following, the invention will be explained in greater detail using an exemplary embodiment explained in the description.

The FIGURES show:

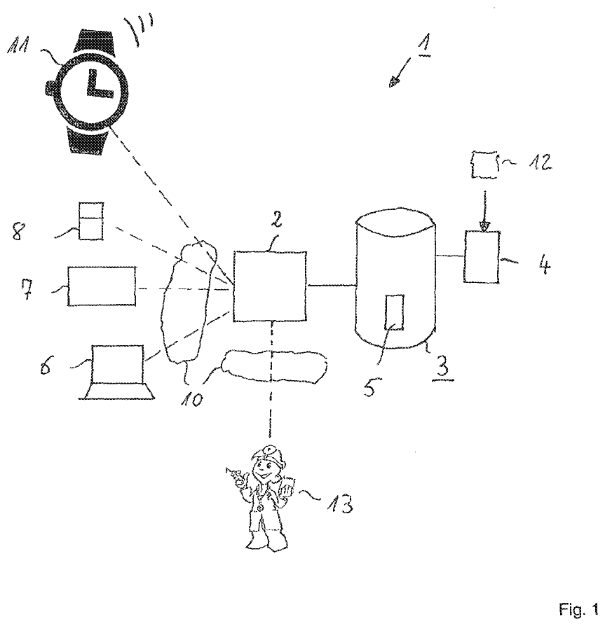

FIG. 1: a sketch-like overview representation of the device according to the invention.

FIG. 1 shows the device 1 according to the invention, for lowering the risk of cancer and/or the risk of a cancer relapse, in a sketch-like representation. According to the representation, the device 1 according to the invention first of all comprises an Internet portal 2, which is connected with a server 3 for operation of the Internet portal 2. In this regard, the server 3 stands in connection, in a data connection, with a connected database 4. Furthermore, diverse comparison and optimization algorithms are located on the server 3.

In this regard, the device 1 according to the invention is supposed to enable or motivate the user of this device 1 to reduce his/her risk of cancer and/or his/her risk of a cancer relapse by means of physical activity, taking the most recent medical knowledge into consideration.

For this purpose, the user, not shown in any detail in the drawing, can enter into a data connection with the server 3, preferably by way of the Internet 10 or other data channels, by way of the Internet portal 2, or by way of various, frequently mobile terminals, such as computers 6, tablets 7, or smart phones 8. For this purpose, the users are usually enabled to enter into a data connection with the Internet portal 2, by means of an application software that can run on the respective terminal 6, 7 or 8. Usually, the users must enter their user data, in other words, in particular, regarding their age, their gender, their weight, their cancer disease, regular data regarding their state of health, possible prior diseases and physical restrictions, which data are relevant for the device 1 according to the invention, into the user portal, by way of a protected data channel, within the scope of use. In this regard, the users can be supported during entry of these data, for example by means of query routines, by way of a suitable interface, as well as by means of possible voice control and other operating aids.

As soon as the users have been registered accordingly, they are supplied with recommendations regarding physical activity by way of the portal 2, taking into consideration and evaluating their data, in such a manner that they ideally develop so much physical activity, ideally every day or at least every week, that regular activation of the enzyme telomerase takes place to such an extent that depending on the scope of physical activity, noticeable lowering or even, in the case of great physical activity, a drastic reduction in the risk of a cancer relapse or the risk of cancer as such is achieved.

The users then have the possibility, vice versa, of entering the physical activities developed by them into the connected database 4 by way of their terminals 6, 7, 8, by way of the portal 2. In this regard, the users are supported during entry of the physical activities by means of corresponding templates stored in the device 1, so that the users merely have to choose between the possible physical activities and, if applicable, merely have to enter a comment regarding the intensity of physical activity, as well as information regarding the time scope of the selected physical activity. Based on these default values, corresponding entry of the physical activity can take place in a few seconds, in each instance. The device 1 therefore offers great ease of use.

In an alternative embodiment, the user can also enter his/her activities into the database 4 by means of independent recording devices, such as a pulse watch 11, for example, which also stands in a data connection with the database 4 by way of the Internet portal 2.

Then, a movement journal is created from the data, which have been entered in user-individual manner, in this regard, by means of an algorithm 5 stored on the server 3, which journal can be called up by the user at any time, by way of his/her respective terminals 6, 7, 8. In particular, the user is given daily information regarding the physical activities performed and, taking these data into consideration, is given a recommendation regarding performance of further physical activities. Furthermore, the user is also informed about the movement dose to be performed on a daily basis, or about the movement dose to be performed on a weekly basis, by means of corresponding numerical displays, for example in percentage information or by means of graphic representations. In this regard, this information serves not only to inform the user but also to motivate him/her. Furthermore, on the basis of stored algorithms 5, the server 3 determines what are called movement equivalents with regard to the physical activities that are still missing for an optimal movement dose, and makes suggestions to the user, particularly also taking into consideration his/her previous user behavior, regarding the physical activities with which he/she can improve or meet his/her daily requirement or weekly requirement.

The respective user is therefore not only given a record of his/her physical activities, but also feedback from the system regarding their success, as well as recommendations as to how this success can be further expanded or perpetuated.

Furthermore, the user has the possibility, to the extent permissible by law, to select a doctor's supervision of his/her physical activities by way of the system. For this purpose, he/she can grant access to his/her user profile data either to a doctor 13 selected by the operator of the portal or to his/her own doctor 13, who then has access to these data and in turn can issue recommendations, taking into consideration his/her medical knowledge, in particular regarding the user, for example regarding prior diseases or personal preferences of the user. In particular, the doctor 13 can also issue corresponding warnings or ask for an appointment in the event that he/she has cause for concern on the basis of the development of the user data. The device 1 according to the invention therefore does not represent a replacement but rather a supplement to a doctor's care for the user.

Furthermore, the device 1 according to the invention is not restricted to a bidirectional data exchange between the Internet portal 2 and the respective user and/or doctor 13. Instead, the users also have the possibility of entering into an exchange with one another, for example in that users publish their movement data, i.e. the data regarding their physical activity, by way of the portal, so that other users have the opportunity to comment on them, express praise or criticism, or to enter into a data exchange with the user, for example so as to come together to perform physical activities jointly. In this regard, the users can choose whether they do this on a public platform, in other words in discussion forums, or on a protected, separate data channel.

This is also a significant motivating component of the device 1 according to the invention.

Ultimately, this aspect also represents a social component. The device 1 according to the invention, in contrast to previously known, usual sports applications, which are predominantly used by persons who are already active in sports and accordingly look for support in their sports activities, also addresses persons who have not previously been active in any sports, are less athletic for reasons of age, or, for example after severe cancer disease, want to reduce their personal cancer risk with the support of the device 1 according to the invention. The latter are often in a difficult personal situation, so that the possible discussion with persons in a similar situation can serve not only for motivation but also for exchange of personal information, for example concerning their personal health history.

In the end result, bringing together the use data relating to the physical activities of the users with reference to their health history, also represents establishment of a relevant database 4 regarding the interaction of physical activities with reference to the risk of cancer or the risk of a cancer relapse, so that with an increasing number of users and by exceeding a statistically relevant minimum size, the database 4 itself can also be evaluated for optimization of the movement recommendations or the recommendations regarding performance of physical activity, something that is done by way of the comparison and optimization algorithms 5 stored on the server 3. In other words, analyses are constantly being performed as to what effects the physical activities performed by the user have on the risk of cancer or the risk of a cancer relapse, wherein in this connection, not only athletic activities but also activities of daily life, such as mowing the lawn, walking up stairs or dusting are recorded and evaluated. Based on these constant evaluations, the recommendations issued to the users with regard to performing physical activities are constantly optimized, so that the device 1 according to the invention can be understood to be a self-learning system in the best sense. For further support of this effect, data 12 from outside the system, such as new medical studies or statistical surveys, can also flow into the database 4 and can furthermore be used to improve the recommendations regarding performance of physical activities or improvement of the device 1 according to the invention as a whole, by way of the comparison and optimization algorithms 5.

Accordingly, the invention relates to a self-learning device 1 for significant reduction of the user-individual risk of cancer or risk of cancer relapse, as well as, depending on the degree of distribution of the device 1 according to the invention, of the risk of cancer or the risk of a cancer relapse as a whole. In the end result, not only the users of the device 1 according to the invention benefit, but rather medical progress as a whole benefits from the implementation of the device 1.

### REFERENCE SYMBOL LIST

1 device
2 Internet portal
3 server
4 database
5 comparison and optimization algorithms
6 computer
7 tablet
8 smart phone
10 Internet
11 pulse watch
12 data from outside the system
13 doctor

1-18. (canceled)

19. A device (1) comprising a detection device for physical activity and/or an input device for manual input of physical activity performed by the user of the device, wherein the detection device and/or the input device is/are data-connected with a server (3), in each instance, with which a database (4) is connected, so that the data recorded by means of the detection device and/or input by means of the input device are transmitted to the database (4) and are automatically displayed on the detection devices and/or input devices of the respective user that are connected with the server (3) of the device (1) and/or can be called up by the respective user by way of a secure data channel, wherein this device (1) can be used for lowering the risk of cancer and/or the risk of a cancer relapse and/or other risks of disease, wherein the enzyme telomerase, i.e. an RNA protein complex having reverse transcriptase activity, is activated, wherein in the database (4), the physical activities recorded or reported by the users are automatically converted to Metabolic Equivalents, abbreviated as METs, by means of a stored concordance table and a conversion formula, to determine the movement dose, and put into relation with an optimal movement dose with reference to a defined period of time, preferably a week, established in user-individualized manner, and converted to a graphic and/or numeric display, wherein the detection device for physical activities is a pulse measurement instrument on an ultrasound basis, and this pulse measurement instrument is implemented in the form of a pulse watch (11), which can preferably be worn on the wrist of the user of the device (1) according to the invention.

20. The device (1) according to claim 19, wherein the input device is a smart phone (8), a tablet (7), a computer (6) or some other unit capable of communicating on the Internet, which is enabled, by means of a mobile app, in other words an application software for mobile operating systems, to enter into a data exchange with the server (3) of the device (1) according to the invention.

21. The device (1) according to claim 19, wherein the data of the users stored in the database (4) are passed to a user-individualized evaluation and furthermore the results of this evaluation are transmitted to a response system connected with the server (3), which system selects suggestions regarding physical activity from a database (4) connected with the server (3), on the basis of this evaluation, and transmits these individual suggestions regarding physical activity to the detection device and/or input device of the respective user that is/are connected with the server (3) of the device (1).

22. The device (1) according to claim 19, wherein a user-individualized movement journal can be drawn up using the database (4) connected with the server (3), which journal can be called up by the respective user at any time, by way of the detection device and/or input device connected with the server (3) of the device (1).

23. The device (1) according to claim 22, wherein the movement journal and/or further profile data of the user in the database (4) can be directly transmitted to a selected doctor (13) and/or called up by this doctor, with the approval of the user.

24. The device (1) according to claim 23, wherein user-individualized data of the doctor (13) selected by the user can be transmitted to the detection device and/or input device of the respective user, which is/are connected with the server (3) of the device (1), by way of the central server (3) of the device (1) according to the invention.

25. The device (1) according to claim 19, wherein in connection with the data indicated for drawing up a user profile, possible physical or other restrictions of the respective user can also be entered into the database (4), and automatic creation of individual movement plans takes place by means of an algorithm (5) connected with the server (3), which algorithm works out a user-individualized movement plan on the basis of the evaluation of the profile data of the user profile and evaluation of the movement journal, from among a plurality of movement plans and/or possible physical activities stored in the system, which plan can then be transmitted to the respective user by way of the detection device and/or input device connected with the server (3) of the device (1) and/or called up by the respective user at any time, and furthermore, this movement plan can be individually adapted as a function of the physical activity of the user, which is transmitted to the server (3) by way of the detection device and/or input device.

26. The device (1) according to claim 19, wherein the users connected with the server (3) of the device (1) by way of the detection device and/or input device can communicate with one another by way of the server (3) and/or can enter into data exchange with one another in discussion forums made available on the server (3) on the portal (2) of the device (1).

27. The device (1) according to claim 19, wherein the user data stored in the database (4) are evaluated in anonymous form, so that a self-learning system is made available by means of comparison and optimization algorithms (5), in such a manner that the effectiveness of the movement plans and/or of the physical activities suggested to the users can be optimized by means of evaluation of the data transmitted by the users, in particular with regard to lowering the risk of cancer and/or the risk of a cancer relapse, and/or constant adaptation of the movement doses and/or physical activities suggested to the users, and calculation and conversion into MET units of the physical activities provided for evaluation takes place by means of entry of new medical knowledge and data (12) into the database (4) of the device (1).

28. The device (1) according to claim 27, wherein user data (12) from outside of the system, for example from medical studies, can be entered into the database (4) and can be used for optimization of the movement plans and/or suggested physical activities and also in the evaluation of the physical activities and their conversion to MET units.

29. The device (1) according to claim 19, wherein an ultrasound head for emission of ultrasonic waves is integrated into the pulse watch (11), wherein the pulse watch (11) can be fastened to the wrist of the respective user of the pulse watch (11) with the interposition of a coupling cushion that follows the underside of the pulse watch (11).

30. The device (1) according to claim 29, wherein the coupling cushion of the pulse watch (11) as such is a gel body or comprises it, wherein the gel body is produced on the basis of reaction products of polyisocyanates and polyols.

31. The device (1) according to claim 29, wherein the gel body of the pulse watch (11) is sheathed with an elastic and/or stretchable sheath, preferably a polymer film.

32. The device (1) according to claim 29, wherein the ultrasound head of the pulse watch (11) is integrated into the gel body.

33. The device (1) according to claim 29, wherein the gel body of the pulse watch (11) is releasably connected with the underside of the pulse watch (11), particularly screwed onto it, with the interposition of a hard plastic.

34. The device (1) according to claim 29, wherein the gel body of the pulse watch (11), with the integrated ultrasound head, is welded into a hard plastic jacket that is releasably connected with the underside of the pulse watch (11).
